(19) United States
(12) Patent Application Publication
(10) Pub. No.: US 2024/0112432 A1
(43) Pub. Date: Apr. 4, 2024

(54) THREE-DIMENSIONAL MODEL GENERATION FOR TUMOR TREATING FIELDS TRANSDUCER LAYOUT

(71) Applicant: Novocure GmbH, Root D4 (CH)

(72) Inventors: Kirill STEPOVOY, Haifa (IL); Reuven Ruby SHAMIR, Haifa (IL); Mor VARDI, Haifa (IL); Gil ZIGELMAN, Haifa (IL); Gidi BAUM, Haifa (IL)

(73) Assignee: Novocure GmbH, Root D4 (CH)

(21) Appl. No.: 18/373,102

(22) Filed: Sep. 26, 2023

Related U.S. Application Data
(60) Provisional application No. 63/411,375, filed on Sep. 29, 2022.

(51) Int. Cl. G06T 19/20; A61B 34/10; G06T 17/20; G16H 50/50

(52) U.S. Cl. CPC G06T 19/20; A61B 34/10; G06T 17/20; G16H 50/50; A61B 2034/105; G06T 2210/41; G06T 2219/2004; G06T 2219/2016

(57) ABSTRACT

A computer-implemented method for generating a three-dimensional (3D) composite model of a region of a subject, the method comprising: generating a 3D clinical model of the region of the subject based on one or more images of the region of the subject; obtaining a 3D generic model of the region of a generic subject; combining the 3D clinical model and the 3D generic model using an affine transformation, a bending transformation, and a squeezing transformation of the 3D generic model to obtain the 3D composite model of the subject; and displaying the composite 3D model on a display.

### CROSS-REFERENCE TO RELATED APPLICATIONS

This application claims priority to U.S. Provisional Application No. 63/411,375, filed Sep. 29, 2022, the entire contents of which are incorporated by reference herein in their entirety.

### BACKGROUND

Tumor treating fields (TTFields) are low intensity alternating electric fields within the intermediate frequency range (for example, 50 kHz to 1 MHz), which may be used to treat tumors as described in U.S. Pat. No. 7,565,205. TTFields are induced non-invasively into a region of interest by transducers placed directly on the subject's body and applying alternating current (AC) voltages between the transducers. Conventionally, a first pair of transducers and a second pair of transducers are placed on the subject's body. AC voltage is applied between the first pair of transducers for a first interval of time to generate an electric field with field lines generally running in the front-back direction. Then, AC voltage is applied at the same frequency between the second pair of transducers for a second interval of time to generate an electric field with field lines generally running in the right-left direction. The system then repeats this two-step sequence throughout the treatment.

### BRIEF DESCRIPTION OF THE DRAWINGS

FIG. 1 is a flowchart depicting an example of generating a three-dimensional (3D) composite model of a region of a subject.

FIG. 2 is a flowchart depicting an example of affine transformation.

FIGS. 3A-3C are examples of a 3D clinical model, a 3D generic model, and a 3D composite model generated according to one embodiment of the disclosed subject matter.

FIG. 4A is an example of displaying transducer array placements on a 3D clinical model of a subject and FIG. 4B is an example of displaying transducer array placements on a 3D composite model of the subject.

FIG. 5 depicts an example computer apparatus for use with the embodiments herein.

Various embodiments are described in detail below with reference to the accompanying drawings, where like reference numerals represent like elements.

### DESCRIPTION OF EMBODIMENTS

To provide a subject with an effective TTFields treatment, precise locations at which to place the transducers on the subject's body must be generated, and these precise locations are based on, for example, the type of the cancer, the size of the cancer, and the location of the cancer in the subject's body. Visualizing the locations on a three-dimensional (3D) model to place the transducers is useful to assist users, e.g., physicians, nurses, assistants, staff members, physicists, dosimetrists, etc., to precisely place the transducers on the subject's body and thus optimizing the tumor treatment. However, generating a 3D model for a subject to use with visualizing transducer locations has certain problems. For example, the scan of the subject may be noisy, resulting in a distorted 3D model of the subject, and some subjects may be uncomfortable viewing a distorted version of their body. As another example, some subjects may be uncomfortable seeing their own body on a display (e.g., the subject's face, or the subject's torso), even if the 3D model is an accurate representation of the subject. As another example, to save cost and/or processing time, only a portion of the subject's body may be scanned (e.g., a partial scan of the subject's head), resulting in a partial 3D model of the subject's body, and some subjects may be uncomfortable seeing such a partial version of their body. The inventors recognized these problems and discovered an approach to generate a 3D composite model of a subject by combining a 3D clinical model of the subject and a 3D generic model that can represent the size, shape, and/or features of the individual subject.

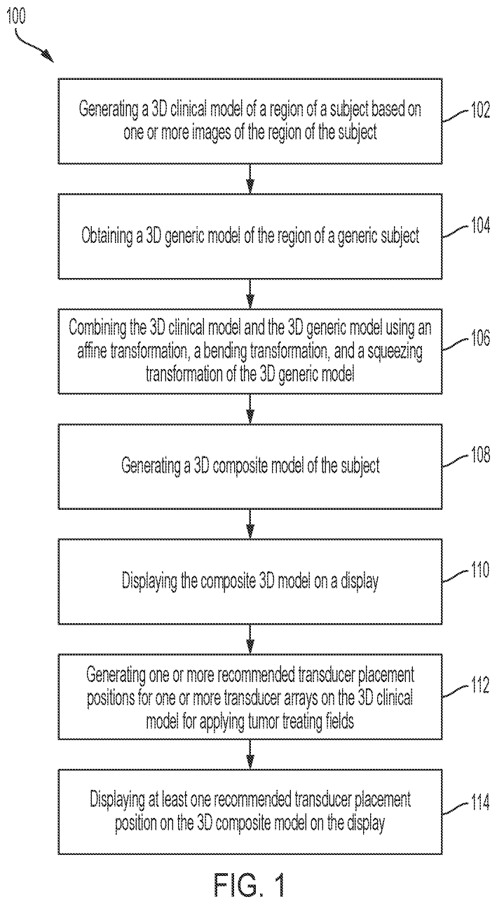

FIG. 1 is a flowchart depicting an example of generating a three-dimensional (3D) composite model of a region of a subject. Certain steps of the method 100 are described as computer-implemented steps. The computer may be any device comprising one or more processors and memory accessible by the one or more processors, the memory storing instructions that when executed by the one or more processors cause the computer to perform the relevant steps of the method 100. While an order of operations is indicated in FIG. 1 for illustrative purposes, the timing and ordering of such operations may vary where appropriate without negating the purpose and advantages of the examples set forth in detail throughout this disclosure.

With reference to FIG. 1, at step 102, the method 100 includes generating a 3D clinical model of a region of the subject based on one or more images of the region of the subject. In some embodiments, the one or more images are medical images. The medical image may, for example, include at least one of a magnetic resonance imaging (MRI) image, a computerized tomography (CT) image, an X-ray image, an ultrasound image, nuclear medicine image, positron-emission tomography (PET) image, arthrogram images, myelogram images, or any image of the subject's body providing an internal view of the subject's body. Each medical image may include an outer shape of a portion of the subject's body and a region corresponding to a region of interest (e.g., tumor) within the subject's body. As an example, the medical image may be a 3D MRI image.

In some embodiments, the image is not limited to medical image and may be any kind of image. In one example, the one or more images are two-dimensional (2D) images that may be captured by one or more user devices. As an example, the one or more user devices may be a cell phone or a camera. In some embodiments, the one or more images include one or more medical images and one or more 2D images captured by one or more user devices.

In some embodiments, the region of the subject includes a region of interest, e.g., a tumor within the subject's body. As an example, the region of the subject is a head of the subject. As an example, the region of the subject is a torso of the subject.

In some embodiments, the 3D clinical model includes a coordinate system. As an example, if the region of the subject includes a head of the subject, the method 100 may further include: identifying a center of the 3D clinical model, where the center is equidistant between a left ear fiducial position and a right ear fiducial position of the 3D clinical model; identifying an X-axis of the 3D clinical model, where the X-axis passes through the center of the 3D clinical model and is between the left ear fiducial position and the right ear fiducial position of the 3D clinical model; identifying a Y-axis of the 3D clinical model, where the Y-axis passes through the center of the 3D clinical model, is orthogonal to the X-axis, and is between a front and a back of the 3D clinical model; and identifying a Z-axis of the 3D clinical model, where the Z-axis passes through the center of the 3D clinical model, is orthogonal to the X-axis and the Y-axis, and is between a top and a bottom of the 3D clinical model. In some embodiments, a surface of the 3D clinical model includes a plurality of meshes.

At step 104, the method 100 includes obtaining a 3D generic model of the region of a generic subject. In some embodiments, a surface of the 3D generic model includes a plurality of meshes. As an example, both the 3D clinical model and the 3D generic model include a coordination system. As an example, if the region of the subject includes a head of the subject, the 3D clinical model and 3D generic model may each include: a center; an X-axis intersecting a left ear fiducial position, a right ear fiducial position, and the center; a Y-axis orthogonal to the X-axis, intersecting the center, and between a front and a back of the head; and a Z-axis orthogonal to the X-axis and the Y-axis and intersecting the center.

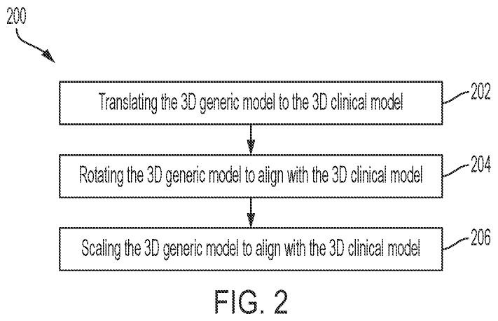

At step 106, the method 100 includes combining the 3D clinical model and the 3D generic model. In some embodiments, the combination of the 3D clinical model and the 3D generic model may be accomplished by using an affine transformation, a bending transformation, and a squeezing transformation of the 3D generic model. As an example, combining the 3D clinical model and the 3D generic model includes deforming the meshes of the 3D generic model in accordance with the meshes the 3D clinical mode. In some embodiments, the method 100 includes transforming the 3D generic model using transformations and the 3D clinical model, where the transformations include an affine transformation, a bending transformation, and a squeezing transformation. Examples of an affine transformation are described further below with respect to FIG. 2.

As for the bending transformation, in some embodiments, if the region of the subject includes a head of the subject, the bending transformation may include transforming an eye location of the 3D generic model to match an eye location of the 3D clinical model without moving ear positions of the 3D generic model. As an example, transforming the eye location of the 3D generic model to match the eye location of the 3D clinical model may include transforming an equidistant point between a left eye fiducial position and a right eye fiducial position of the 3D generic model to align with an equidistant point between a left eye fiducial position and a right eye fiducial position of the 3D clinical model. In some embodiments, the bending transformation of the 3D generic model may include bending the 3D generic model in accordance with the 3D clinical model at the X-axis, where after bending the 3D generic model, a front position of the 3D generic model is on the Y-axis, where the front position of the 3D generic model is a position equidistant between a left eye fiducial position and a right eye fiducial position of the 3D generic model. In some embodiments, the bending transformation is a second order transformation.

As for the squeezing transformation, in some embodiments, the squeezing transformation may include transforming the 3D generic model to match the 3D clinical model. In some embodiments, squeezing transformation of the 3D generic model includes squeezing the 3D generic model in accordance with the 3D clinical model at the X-axis. In some embodiments, the squeezing transformation is a second order transformation.

At step 108, the method 100 includes generating a 3D composite model of the subject based on combination of the 3D clinical model and the 3D generic model at step 106. As an example, the method 100 may further include performing surface fitting on the 3D composite model, where the surface fitting procedure includes at least one of interpolation or extrapolation.

At step 110, the method includes displaying the 3D composite model on a display. In some embodiments, the display is on a user interface. As an example, a user may select to display the 3D clinical model and the 3D composite model on the display for comparison.

At step 112, the method 100 includes generating one or more recommended transducer placement positions for one or more transducer arrays on the 3D clinical model for applying tumor treating fields. In some embodiments, the one or more recommended transducer placement positions are generated based on, for example, the region of interest of the subject's body corresponding to the tumor. As an example, the one or more recommended transducer placement positions may be intended to optimize tumor treatment dose delivered to the region of interest of the subject's body. In some embodiments, the one or more recommended transducer placement positions may be generated based on the 3D composite model.

At step 114, the method includes displaying at least one recommended transducer placement position on the 3D composite model on the display. Examples of generating and displaying one or more recommended transducer placement positions are illustrated in FIGS. 4A and 4B, which are discussed further below.

FIG. 2 is a flowchart depicting an example of an affine transformation. Certain steps of the method 200 are described as computer-implemented steps. The computer may be any device comprising one or more processors and memory accessible by the one or more processors, the memory storing instructions that when executed by the one or more processors cause the computer to perform the relevant steps of the method 200. While an order of operations is indicated in FIG. 2 for illustrative purposes, the timing and ordering of such operations may vary where appropriate without negating the purpose and advantages of the examples set forth in detail throughout this disclosure.

At step 202, the method 200 includes translating the 3D generic model to the 3D clinical model. In some embodiments, the translation of the 3D generic model to the 3D clinical model includes: identifying a center of the 3D clinical model (as an example, if the region of the subject includes a head of the subject, the center may be equidistant between a left ear fiducial position and a right ear fiducial position of the 3D clinical model); identifying a center of the 3D generic model (as an example, if the region of the subject includes a head of the subject, the center may be equidistant between a left ear fiducial position and a right ear fiducial position of the 3D generic model); and translating the 3D generic model so that the center of the 3D generic model overlaps the center of the 3D clinical model.

At step 204, the method 200 includes rotating the 3D generic model to align with the 3D clinical model. In some embodiments, the rotation of the 3D generic model to align with the 3D clinical model may include identifying a first location of the 3D clinical model; identifying a first location of the 3D generic model corresponding to the first location of the 3D clinical model; and rotating the 3D generic model so that the first location of the 3D generic model overlaps the first location of the 3D clinical model. In some embodiments, if the region of the subject includes a head of the subject, the rotation of the 3D generic model to align with the 3D clinical model may include identifying an eye location of the 3D clinical model; identifying an eye location of the 3D generic model; and rotating the 3D generic model so that the eye location of the 3D generic model overlaps the eye location of the 3D clinical model. As an example, the eye location of the 3D clinical model may be equidistant between a left eye fiducial position and a right eye fiducial position of the 3D clinical model, and the eye location of the 3D generic model may be equidistant between a left eye fiducial position and a right eye fiducial position of the 3D generic model.

In some embodiments, as discussed in FIG. 1, both the 3D clinical model and the 3D generic model include a coordination system. In some embodiments, rotation of the 3D generic model to align with the 3D clinical model may include rotating the 3D generic model around the X-axis to align a first location of the 3D generic model with a corresponding first location of the 3D clinical model on a same x-y plane. As an example, if the region of the subject includes a head of the subject, rotation of the 3D generic model to align with the 3D clinical model may include rotating the 3D generic model around the X-axis to align the eye location of the 3D generic model with the eye location of the 3D clinical model on a same x-y plane.

At step 206, the method 200 includes scaling the 3D generic model to align with the 3D clinical model. In some embodiments, scaling of the 3D generic model to align with the 3D clinical model may include: scaling the 3D generic model so that a first region of the 3D generic model aligns with a corresponding first region of the 3D clinical model (for example, if the region of the subject includes a head of the subject, so that an ear region of the 3D generic model aligns with an ear region of the 3D clinical model); and scaling the 3D generic model so that a second region of the 3D generic model aligns with a corresponding second region of the 3D clinical model (for example, if the region of the subject includes a head of the subject, so that an eye region of the 3D generic model aligns with an eye region of the 3D clinical model). As an example, a distance between a left ear fiducial position and a right ear fiducial position of the 3D generic model may be scaled to match a distance between a left ear fiducial position and a right ear fiducial position of the 3D clinical model, and a distance between a left eye fiducial position and a right eye fiducial position of the 3D generic model is scaled to match a distance between a left eye fiducial position and a right eye fiducial position of the 3D clinical model.

In some embodiments, as discussed above, both of the 3D clinical model and the 3D generic model include a coordination system. In some embodiments, scaling of the 3D generic model to align with the 3D clinical model may include: scaling the X-axis of the 3D generic model so that a distance between two locations on the 3D generic model is the same as a distance between two corresponding locations on the 3D clinical model (for example, if the region of the subject includes a head of the subject, so that a distance between ears on the 3D generic model is the same as a distance between ears on the 3D clinical model); and scaling the Y-axis of the 3D generic model so that a distance between a first location and the center of the 3D generic model is the same as a distance between a corresponding first location and the center on the 3D clinical model (for example, if the region of the subject includes a head of the subject, so that a distance between an eye location and the center of the 3D generic model is the same as a distance between an eye location and the center on the 3D clinical model). As an example, the distance between the ears on the 3D clinical model may be a distance between a left ear fiducial position and a right ear fiducial position of the 3D clinical model, and the distance between the eye location and the center on the 3D clinical model may be a distance between a left eye fiducial position and a right eye fiducial position of the 3D clinical model.

In some embodiments, scaling of the 3D generic model to align with the 3D clinical model may include scaling the 3D generic model in accordance with the 3D clinical model at the X-axis, Y-axis, and Z-axis. As an example, scaling the X-axis of the 3D generic model may include: setting a distance between left and right positions of the 3D generic model to be the same as a distance between corresponding left and right positions of the clinical 3D model (for example, if the region of the subject includes a head of the subject, setting a distance between left and right ear fiducial positions of the 3D generic model to be the same as a distance between left and right ear fiducial positions of the clinical 3D head model); scaling the Y-axis of the 3D generic model may include setting a distance between a front position and the center of the 3D generic model to be the same as a distance between a front position and the center of the 3D clinical model; and scaling the Z-axis of the 3D generic model may include scaling the Z-axis with the same scaling as the X-axis. As an example, the front position of the 3D generic model may be a position equidistant between a left eye fiducial position and a right eye fiducial position of the 3D generic model, and the front position of the 3D clinical model may be a position equidistant between a left eye fiducial position and a right eye fiducial position of the 3D clinical model.

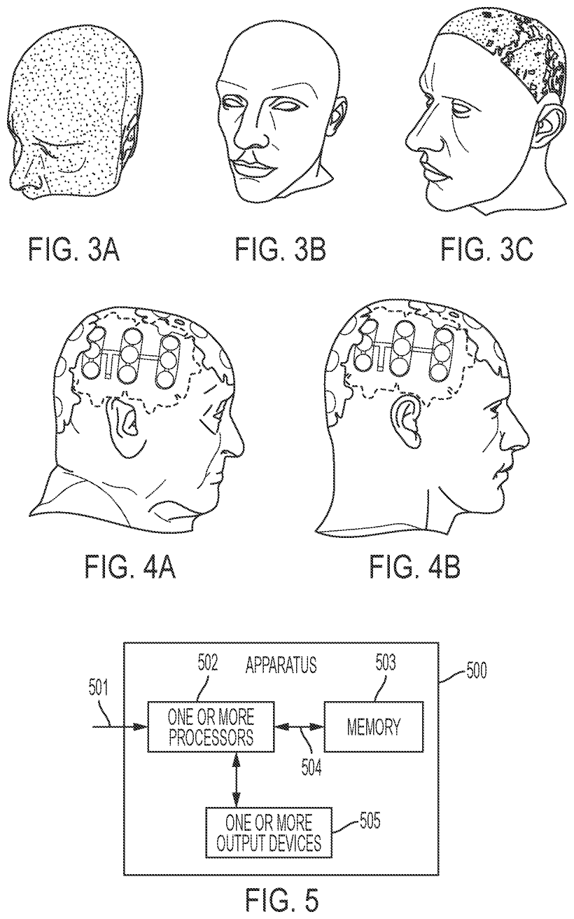

FIGS. 3A-3C are examples of a 3D clinical model, a 3D generic model, and a 3D composite model generated according to an exemplary embodiment. In the example depicted in FIG. 3A, a 3D clinical model of a region of the subject (e.g., head of the subject) is generated based on one or more images of the region of the subject. As shown in FIG. 3A, the 3D clinical model represents the shape, size, features, etc. of the subject's head. However, the 3D clinical model has noise, e.g., the eyes and ears of the model are not clearly shown. In addition, the 3D clinical model is a partial version of the subject's head. FIG. 3B is an example of 3D generic model of the region, e.g., head. FIG. 3C is a 3D composite model of the subject based on the combination of the 3D clinical model in FIG. 3A and the 3D generic model in FIG. 3B. As shown in FIG. 3C, the 3D composite model represents the shape, size, feature, etc. of the subject's head, has little noise, e.g., the eyes and ears of the model are clearly shown, and is a full version, and not a partial version, of the subject's body part, e.g., head of the subject.

FIG. 4A is an example of displaying transducer array placements on a 3D clinical model of a subject. As an example, the transducer array placement is one of recommended transducer array positions for applying tumor treating fields generated at step 112 of FIG. 1. FIG. 4B is an example of displaying transducer array placements on a 3D composite model of the subject. Although FIGS. 4A and 4B illustrate transducer arrays having circular-shaped electrode elements, the electrode elements may have a variety of shapes.

FIG. 5 depicts an example computer apparatus for use with the embodiments herein. As an example, the apparatus 500 may be a computer to implement certain inventive techniques disclosed herein. For example, the methods of FIGS. 1 and 2 may be performed by a computer apparatus, such as apparatus 500. The apparatus 500 may include one or more processors 502, memory 503, one or more input devices, and one or more output devices 505.

In one example, based on input 501, the one or more processors generate a 3D composite model according to embodiments herein. In one example, the input 501 is user input. In another example, the input 501 is one or more images of a region of the subject. In another example, the input 501 may be from another computer in communication with the apparatus 500. The input 501 may be received in conjunction with one or more input devices (not shown) of the apparatus 500.

The memory 503 may be accessible by the one or more processors 502 (e.g., via a link 504) so that the one or more processors 502 can read information from and write information to the memory 503. The memory 503 may store instructions that when executed by the one or more processors 502 implement one or more embodiments described herein. The memory 503 may be a non-transitory computer readable medium (or a non-transitory processor readable medium) containing a set of instructions thereon for generating a 3D composite model of a region of a subject, wherein when executed by a processor (such as one or more processors 502), the instructions cause the processor to perform one or more methods disclosed herein.

The one or more output devices 505 may provide the status of the computer-implemented techniques herein. The one or more output devices 505 may provide visualization data according to certain embodiments of the invention, such as the medical image, 3D clinical model, 3D generic model, 3D composite model, and/or transducer placements on the 3D composite model. The one or more output devices 505 may include one or more displays, e.g., monitors, liquid crystal displays, organic light-emitting diode displays, active-matrix organic light-emitting diode displays, stereo displays, etc.

The apparatus 500 may be an apparatus for generating a 3D composite model of a region of a subject, the apparatus including: one or more processors (such as one or more processors 502); and memory (such as memory 503) accessible by the one or more processors, the memory storing instructions that when executed by the one or more processors, cause the apparatus to perform one or more methods disclosed herein.

### ILLUSTRATIVE EMBODIMENTS

The invention includes other illustrative embodiments, such as the following.

Illustrative Embodiment 1. A computer-implemented method for generating a three-dimensional (3D) composite model of a region of a subject, the method comprising: generating a 3D clinical model of the region of the subject based on one or more images of the region of the subject; obtaining a 3D generic model of the region of a generic subject; combining the 3D clinical model and the 3D generic model using an affine transformation, a bending transformation, and a squeezing transformation of the 3D generic model to obtain the 3D composite model of the subject; and displaying the composite 3D model on a display.

Illustrative Embodiment 2. The computer-implemented method of Illustrative Embodiment 1, wherein the affine transformation comprises: translating the 3D generic model to the 3D clinical model; rotating the 3D generic model to align with the 3D clinical model; and scaling the 3D generic model to align with the 3D clinical model.

Illustrative Embodiment 3. The computer-implemented method of Illustrative Embodiment 2, wherein translating the 3D generic model to the 3D clinical model comprises: identifying a center of the 3D clinical model; identifying a center of the 3D generic model; and translating the 3D generic model so that the center of the 3D generic model overlaps the center of the 3D clinical model.

Illustrative Embodiment 4. The computer-implemented method of Illustrative Embodiment 3, wherein the center of the 3D clinical model is equidistant between a left ear fiducial position and a right ear fiducial position of the 3D clinical model.

Illustrative Embodiment 5. The computer-implemented method of Illustrative Embodiment 2, wherein rotating the 3D generic model to align with the 3D clinical model comprises: identifying an eye location of the 3D clinical model; identifying an eye location of the 3D generic model; and rotating the 3D generic model so that the eye location of the 3D generic model overlaps the eye location of the 3D clinical model.

Illustrative Embodiment 6. The computer-implemented method of Illustrative Embodiment 5, wherein the eye location of the 3D clinical model is equidistant between a left eye fiducial position and a right eye fiducial position of the 3D clinical model.

Illustrative Embodiment 7. The computer-implemented method of Illustrative Embodiment 2, wherein scaling the 3D generic model to align with the 3D clinical model comprises: scaling the 3D generic model so that an ear region of the 3D generic model aligns with an ear region of the 3D clinical model; and scaling the 3D generic model so that an eye region of the 3D generic model aligns with an eye region of the 3D clinical model.

Illustrative Embodiment 8. The computer-implemented method of Illustrative Embodiment 7, wherein a distance between a left ear fiducial position and a right ear fiducial position of the 3D generic model is scaled to match a distance between a left ear fiducial position and a right ear fiducial position of the 3D clinical model, and wherein a distance between a left eye fiducial position and a right eye fiducial position of the 3D generic model is scaled to match a distance between a left eye fiducial position and a right eye fiducial position of the 3D clinical model.

Illustrative Embodiment 9. The computer-implemented method of Illustrative Embodiment 1, wherein the bending transformation comprises: transforming an eye location of the 3D generic model to match an eye location of the 3D clinical model without moving ear positions of the 3D generic model.

Illustrative Embodiment 10. The computer-implemented method of Illustrative Embodiment 9, wherein an equidistant point between a left eye fiducial position and a right eye fiducial position of the 3D generic model is transformed to align with an equidistant point between a left eye fiducial position and a right eye fiducial position of the 3D clinical model.

Illustrative Embodiment 11. The computer-implemented method of Illustrative Embodiment 9, wherein the bending transformation is a second order transformation.

Illustrative Embodiment 12. The computer-implemented method of Illustrative Embodiment 1, wherein the squeezing transformation comprises: transforming the 3D generic model to match the 3D clinical model.

Illustrative Embodiment 13. The computer-implemented method of Illustrative Embodiment 12, wherein the squeezing transformation is a second order transformation.

Illustrative Embodiment 14. The computer-implemented method of Illustrative Embodiment 1, further comprising: performing surface fitting on the 3D composite model, wherein the surface fitting procedure comprises at least one of interpolation or extrapolation.

Illustrative Embodiment 15. The computer-implemented method of Illustrative Embodiment 1, further comprising: generating one or more recommended transducer array positions for one or more transducer arrays on the 3D clinical model for applying tumor treating fields; and displaying at least one of the recommended transducer array positions on the 3D composite model on the display.

Illustrative Embodiment 16. The computer-implemented method of Illustrative Embodiment 1, wherein the region of the subject is a head of the subject.

Illustrative Embodiment 17. The computer-implemented method of Illustrative Embodiment 1, wherein the region of the subject is a torso of the subject.

Illustrative Embodiment 18. The computer-implemented method of Illustrative Embodiment 1, further comprising: identifying a center of the 3D clinical model, wherein the center is equidistant between a left ear fiducial position and a right ear fiducial position of the 3D clinical model; identifying an X-axis of the 3D clinical model, wherein the X-axis passes through the center of the 3D clinical model and is between the left ear fiducial position and the right ear fiducial position of the 3D clinical model; identifying a Y-axis of the 3D clinical model, wherein the Y-axis passes through the center of the 3D clinical model, is orthogonal to the X-axis, and is between a front and a back of the 3D clinical model; and identifying a Z-axis of the 3D clinical model, wherein the Z-axis passes through the center of the 3D clinical model, is orthogonal to the X-axis and the Y-axis, and is between a top and a bottom of the 3D clinical model.

Illustrative Embodiment 19. The computer-implemented method of Illustrative Embodiment 18, wherein the affine transformation comprises: rotating the 3D generic model around the X-axis to align an eye location of the 3D generic model with an eye location of the 3D clinical model on a same x-y plane.

Illustrative Embodiment 20. The computer-implemented method of Illustrative Embodiment 19, wherein the eye location of the 3D clinical model is equidistant between a left eye fiducial position and a right eye fiducial position of the 3D clinical model.

Illustrative Embodiment 21. The computer-implemented method of Illustrative Embodiment 18, wherein the affine transformation comprises: scaling the X-axis of the 3D generic model so that a distance between ears on the 3D generic model is the same as a distance between ears on the 3D clinical model; and scaling the Y-axis of the 3D generic model so that a distance between an eye location and the center of the 3D generic model is the same as a distance between an eye location and the center on the 3D clinical model.

Illustrative Embodiment 22. The computer-implemented method of Illustrative Embodiment 21, wherein the distance between the ears on the 3D clinical model is a distance between a left ear fiducial position and a right ear fiducial position of the 3D clinical model, and wherein the distance between the eye location and the center on the 3D clinical model is a distance between a left eye fiducial position and a right eye fiducial position of the 3D clinical model.

Illustrative Embodiment 23. An apparatus to generate a three-dimensional (3D) composite model of a head of a subject, the apparatus comprising: one or more processors; and memory accessible by the one or more processors, the memory storing instructions that when executed by the one or more processors, cause the apparatus to: generate a 3D clinical model of the head of the subject based on one or more images of the head of the subject; obtain a 3D generic model of a head of a generic subject; transform the 3D generic model using transformations and the 3D clinical model, wherein the transformations comprise an affine transformation, a bending transformation, and a squeezing transformation; generate the 3D composite model based on the transformed 3D generic model and the 3D clinical model; and display the 3D composite model on a display.

Illustrative Embodiment 24. The apparatus of Illustrative Embodiment 23, wherein the 3D clinical model and the 3D generic model each comprise: a center; an X-axis intersecting a left ear fiducial position, a right ear fiducial position, and the center; a Y-axis orthogonal to the X-axis, intersecting the center, and between a front and a back of the head; and a Z-axis orthogonal to the X-axis and the Y-axis and intersecting the center.

Illustrative Embodiment 25. The apparatus of Illustrative Embodiment 24, wherein the affine transformation of the 3D generic model comprises: overlapping the center of the 3D generic model with the center of the 3D clinical model; and rotating the 3D generic model around the X-axis to place a position equidistant between a left eye fiducial position and a right eye fiducial position of the 3D generic model on an x-y plane.

Illustrative Embodiment 26. The apparatus of Illustrative Embodiment 24, wherein the affine transformation of the 3D generic model comprises: scaling the 3D generic model in accordance with the 3D clinical model at the X-axis, Y-axis, and Z-axis, wherein scaling the X-axis of the 3D generic model comprises setting a distance between left and right ear fiducial positions of the 3D generic model to be the same as a distance between left and right ear fiducial positions of the clinical 3D head model, wherein scaling the Y-axis of the 3D generic model comprises setting a distance between a front position and the center of the 3D generic model to be the same as a distance between a front position and the center of the 3D clinical model, wherein the front position of the 3D generic model is a position equidistant between a left eye fiducial position and a right eye fiducial position of the 3D generic model, wherein the front position of the 3D clinical model is a position equidistant between a left eye fiducial position and a right eye fiducial position of the 3D clinical model; and wherein scaling the Z-axis of the 3D generic model comprises scaling the Z-axis with the same scaling as the X-axis.

Illustrative Embodiment 27. The apparatus of Illustrative Embodiment 24, wherein the bending transformation of the 3D generic model comprises: bending the 3D generic model in accordance with the 3D clinical model at the X-axis, wherein after bending the 3D generic model, a front position of the 3D generic model is on the Y-axis, wherein the front position of the 3D generic model is a position equidistant between a left eye fiducial position and a right eye fiducial position of the 3D generic model.

Illustrative Embodiment 28. The apparatus of Illustrative Embodiment 24, wherein the squeezing transformation of the 3D generic model comprises: squeezing the 3D generic model in accordance with the 3D clinical model at the X-axis.

Illustrative Embodiment 29. A non-transitory computer-readable medium comprising instructions to generate one or more recommended transducer placement positions on a subject, the instructions when executed by a computer cause the computer to perform a method comprising: generating a 3D clinical model of the subject based on one or more images of the subject; obtaining a 3D generic model of a generic subject; combining the 3D clinical model and the 3D generic model using an affine transformation, a bending transformation, and a squeezing transformation of the 3D generic model to obtain a 3D composite model of the subject; and generating one or more recommended transducer placement positions for one or more transducer arrays on the 3D clinical model for applying tumor treating fields; displaying at least one recommended transducer placement position on the 3D composite model on a display.

Illustrative Embodiment 30. The non-transitory computer-readable medium of Illustrative Embodiment 29, wherein a surface of the 3D clinical model comprises a plurality of meshes, wherein a surface of the 3D generic model comprises a plurality of meshes, wherein combining the 3D clinical model and the 3D generic model comprises deforming the meshes of the 3D generic model in accordance with the meshes the 3D clinical model.

Illustrative Embodiment 31. The non-transitory computer-readable medium of Illustrative Embodiment 29, wherein combining the 3D clinical model and the 3D generic model comprises using an affine transformation, a bending transformation, and a squeezing transformation of 3D generic model.

What is claimed is:

1. A computer-implemented method for generating a three-dimensional (3D) composite model of a region of a subject, the method comprising:
generating a 3D clinical model of the region of the subject based on one or more images of the region of the subject;
obtaining a 3D generic model of the region of a generic subject;
combining the 3D clinical model and the 3D generic model using an affine transformation, a bending transformation, and a squeezing transformation of the 3D generic model to obtain the 3D composite model of the subject; and
displaying the composite 3D model on a display.

2. The computer-implemented method of claim 1, wherein the affine transformation comprises:
translating the 3D generic model to the 3D clinical model;
rotating the 3D generic model to align with the 3D clinical model; and
scaling the 3D generic model to align with the 3D clinical model.

3. The computer-implemented method of claim 2, wherein translating the 3D generic model to the 3D clinical model comprises:
identifying a center of the 3D clinical model;
identifying a center of the 3D generic model; and
translating the 3D generic model so that the center of the 3D generic model overlaps the center of the 3D clinical model.

4. The computer-implemented method of claim 2, wherein rotating the 3D generic model to align with the 3D clinical model comprises:
identifying an eye location of the 3D clinical model;
identifying an eye location of the 3D generic model; and
rotating the 3D generic model so that the eye location of the 3D generic model overlaps the eye location of the 3D clinical model.

5. The computer-implemented method of claim 2, wherein scaling the 3D generic model to align with the 3D clinical model comprises:
scaling the 3D generic model so that an ear region of the 3D generic model aligns with an ear region of the 3D clinical model; and
scaling the 3D generic model so that an eye region of the 3D generic model aligns with an eye region of the 3D clinical model.

6. The computer-implemented method of claim 1, wherein the bending transformation comprises:
transforming an eye location of the 3D generic model to match an eye location of the 3D clinical model without moving ear positions of the 3D generic model.

7. The computer-implemented method of claim 1, wherein the squeezing transformation comprises:
transforming the 3D generic model to match the 3D clinical model.

8. The computer-implemented method of claim 1, further comprising:
performing surface fitting on the 3D composite model, wherein the surface fitting procedure comprises at least one of interpolation or extrapolation.

9. The computer-implemented method of claim 1, further comprising:
generating one or more recommended transducer array positions for one or more transducer arrays on the 3D clinical model for applying tumor treating fields; and
displaying at least one of the recommended transducer array positions on the 3D composite model on the display.

10. The computer-implemented method of claim 1, wherein the region of the subject is a head of the subject.

11. The computer-implemented method of claim 1, wherein the region of the subject is a torso of the subject.

12. An apparatus to generate a three-dimensional (3D) composite model of a head of a subject, the apparatus comprising: one or more processors; and memory accessible by the one or more processors, the memory storing instructions that when executed by the one or more processors, cause the apparatus to:
generate a 3D clinical model of the head of the subject based on one or more images of the head of the subject;
obtain a 3D generic model of a head of a generic subject;
transform the 3D generic model using transformations and the 3D clinical model, wherein the transformations comprise an affine transformation, a bending transformation, and a squeezing transformation;
generate the 3D composite model based on the transformed 3D generic model and the 3D clinical model; and
display the 3D composite model on a display.

13. The apparatus of claim 12, wherein the 3D clinical model and the 3D generic model each comprise:
a center;
an X-axis intersecting a left ear fiducial position, a right ear fiducial position, and the center;
a Y-axis orthogonal to the X-axis, intersecting the center, and between a front and a back of the head; and
a Z-axis orthogonal to the X-axis and the Y-axis and intersecting the center.

14. The apparatus of claim 13, wherein the affine transformation of the 3D generic model comprises:
overlapping the center of the 3D generic model with the center of the 3D clinical model; and
rotating the 3D generic model around the X-axis to place a position equidistant between a left eye fiducial position and a right eye fiducial position of the 3D generic model on an x-y plane.

15. The apparatus of claim 13, wherein the affine transformation of the 3D generic model comprises:
scaling the 3D generic model in accordance with the 3D clinical model at the X-axis, Y-axis, and Z-axis,
wherein scaling the X-axis of the 3D generic model comprises setting a distance between left and right ear fiducial positions of the 3D generic model to be the same as a distance between left and right ear fiducial positions of the clinical 3D head model,
wherein scaling the Y-axis of the 3D generic model comprises setting a distance between a front position and the center of the 3D generic model to be the same as a distance between a front position and the center of the 3D clinical model,
wherein the front position of the 3D generic model is a position equidistant between a left eye fiducial position and a right eye fiducial position of the 3D generic model,
wherein the front position of the 3D clinical model is a position equidistant between a left eye fiducial position and a right eye fiducial position of the 3D clinical model; and
wherein scaling the Z-axis of the 3D generic model comprises scaling the Z-axis with the same scaling as the X-axis.

16. The apparatus of claim 13, wherein the bending transformation of the 3D generic model comprises:
bending the 3D generic model in accordance with the 3D clinical model at the X-axis, wherein after bending the 3D generic model, a front position of the 3D generic model is on the Y-axis,
wherein the front position of the 3D generic model is a position equidistant between a left eye fiducial position and a right eye fiducial position of the 3D generic model.

17. The apparatus of claim 13, wherein the squeezing transformation of the 3D generic model comprises:
squeezing the 3D generic model in accordance with the 3D clinical model at the X-axis.

18. A non-transitory computer-readable medium comprising instructions to generate one or more recommended transducer placement positions on a subject, the instructions when executed by a computer cause the computer to perform a method comprising:
generating a 3D clinical model of the subject based on one or more images of the subject;
obtaining a 3D generic model of a generic subject;
combining the 3D clinical model and the 3D generic model using an affine transformation, a bending transformation, and a squeezing transformation of the 3D generic model to obtain a 3D composite model of the subject; and
generating one or more recommended transducer placement positions for one or more transducer arrays on the 3D clinical model for applying tumor treating fields;
displaying at least one recommended transducer placement position on the 3D composite model on a display.

19. The non-transitory computer-readable medium of claim 18, wherein a surface of the 3D clinical model comprises a plurality of meshes,
wherein a surface of the 3D generic model comprises a plurality of meshes,
wherein combining the 3D clinical model and the 3D generic model comprises deforming the meshes of the 3D generic model in accordance with the meshes the 3D clinical model.

20. The non-transitory computer-readable medium of claim 18, wherein combining the 3D clinical model and the 3D generic model comprises using an affine transformation, a bending transformation, and a squeezing transformation of 3D generic model.
